(19) United States
(12) Patent Application Publication
(10) Pub. No.: US 2019/0343642 A1
(43) Pub. Date: Nov. 14, 2019

(54) MENISCUS PROSTHESIS

(71) Applicant: Atro Medical B.V., Nijmegen (NL)

(72) Inventors: Jacob Koenen, Sittard (NL); Edwin Daamen, Born (NL); Tony van Tienen, Nijmegen (NL); Pieter Buma, Nijmegen (NL)

(73) Assignee: Atro Medical B.V., Nijmegen (NL)

(21) Appl. No.: 16/504,414

(22) Filed: Jul. 8, 2019

Related U.S. Application Data
(63) Continuation-in-part of application No. 15/124,412, filed on Sep. 8, 2016.

(30) Foreign Application Priority Data
Mar. 11, 2014 (EP) 14158887.1

(51) Int. Cl. A61F 2/38

(52) U.S. Cl. CPC A61F 2/3872; A61F 2002/30014

(57) ABSTRACT

A meniscus prosthesis includes a core made of a first biocompatible, non-resorbable material having a first tensile modulus. The core includes an arc-shaped body having: a first end having a first through-hole; a second end having a second through-hole; a curved intermediate section connecting the first end and the second end; a first surface configured to face a first interior surface of the joint during use and a second surface configured to face a second interior surface of the joint during use, an inner edge and an outer edge. The core comprises a transverse cross-section in which the width is greater than the height along the length of the core. A cushioning material surrounds the intermediate section of the core, the cushioning material being made of a second biocompatible, non-resorbable material having a second tensile modulus, which is lower than a tensile modulus of the first material.

### CROSS-REFERENCE TO RELATED APPLICATIONS

This application is a continuation in part of U.S. patent application Ser. No. 15/124,412, which is the US national phase of International Application PCT/EP2015/054906, filed 10 Mar. 2015, and claiming priority to European patent application EP14158887.1, filed 11 Mar. 2014. Each of the aforementioned applications is hereby incorporated by reference in its entirety.

### FIELD

The invention is directed to a meniscus prosthesis, a process for the production of the meniscus prosthesis and a method for replacing a native meniscus by the meniscus prosthesis.

### BACKGROUND

The meniscus distributes loads from the femur to the tibia plateau and by its adaptation to the contours of the joint, together with its low friction surface, it provides a smooth nearly frictionless motion of the knee joint. The highly oriented circumferential and radial collagen bundles make the matrix of the meniscus highly anisotropic. Tears (damages) can occur in the meniscus, causing pain and function loss of the knee joint. When tears occur in the meniscus generally a part of the meniscus tissue or the meniscus itself has to be removed. Removal of meniscus tissue may lead to serious osteoarthritic degeneration of the knee joint, especially when a (sub)total meniscectomy was necessary. A meniscus prosthesis would postpone or even prevent other extensive and expensive knee surgeries, such as a total knee replacement. By replacing the ectomized meniscus by an artificial implant the normal joint homeostasis would be restored, the pain could diminish, the function could be restored and further osteoarthritic degeneration could be prevented. Likely this would reduce the cost of healthcare since the number of expensive joint replacement procedures would be reduced.

Meniscus prostheses are known in the prior art. For example, WO2008/127942 describes a human implantable meniscus device with an anchoring system for locking the device into a bone. Surgically drilled bore channels in the tibial plateau are needed to lock the device. The device is made of a flexible and resilient material

WO2012/168715 describes an implant system for implantation at a joint including an implant device. The implant device comprises an elongate member and a fixation device attached to a body portion. To fix the implant system in the knee joint the fixation device is attached to the tibia by a staple or a screw. For fixation of the elongate member a large channel has to be provided in the tibia bone. The body portion of the implant device comprises a reinforcement structure that is embedded within an elastomeric polymer.

In WO2011/138045 a non-resorbable meniscus prosthesis is described. The non-resorbable meniscus comprises bone plugs and/or sutures for the fixation of the meniscus prosthesis in the knee joint. A disadvantage of the meniscus prosthesis described in WO2011/138045 is that it takes a relatively long time before the bone plugs are permanently attached due to the relatively slow osseous ingrowth. The body of the meniscus prosthesis is made of one type of biocompatible material.

US20130131805 describes an orthopaedic implant comprising different distinct sections, wherein each section comprises a different polymeric material. The orthopaedic implant can be a meniscus implant. The polymeric material preferably is a polyurethane block copolymer.

In WO2008/045807, a meniscus prosthetic device is described comprising a body portion and a fixation member. The body portion and the fixation member form a monolithic structure comprising a flexible polymeric material; preferably a polyurethane. The body portion can comprise a deformation control element comprising a material having increased stiffness relative to the material of the body portion.

### SUMMARY

It is an object of the present invention to provide a meniscus prosthesis for the human knee joint with an improved shape and improved mechanical properties, which is easy to implant in the knee joint. This object may be achieved by providing a meniscus prosthesis comprising two parts, each configured to perform a different function: a load divider, configured to distribute the vertical load from the condyle to the tibial plateau, and a retainer, configured to withstand the hoop stress through the crescent shaped prosthesis, which results from the rounded shape of the condyle.

This object is achieved by a meniscus prosthesis as described below.

In a first aspect, there is provided a meniscus prosthesis comprising: a core made of a first biocompatible, non-resorbable material having a first tensile modulus, wherein the core comprises an arc-shaped body. The arc shaped body extends from a first end, having a first through-hole configured to receive an anchor for securing the first end to a bone surface, to a second end having a second through-hole configured to receive an anchor for securing the second end to a bone surface. A curved intermediate section connects the first end and the second end. The core further comprises a first surface configured to face a first interior surface of the joint during use and a second surface configured to face a second interior surface of the joint during use, wherein the first and second through-holes extend from the first surface to the second surface. The arc shaped body also comprises an inner edge and an outer edge, wherein the inner edge is located on the concave side of the curve, whilst the outer edge is located on the convex edge of the curve. A width W is defined between the inner edge and the outer edge of the core, and wherein a maximum height Hₘₐₓ is defined, perpendicular to the width W, between the first surface and the second surface. The core is configured such that it comprises a transverse cross-section in which W is greater than H, along the length of the core.

A cushioning material surrounds or covers at least the intermediate section of the core, the cushioning material being made of a second biocompatible, non-resorbable material having a second tensile modulus, which is lower than a tensile modulus of the first material.

In at least one embodiment, a difference between the first tensile modulus and the second tensile modulus is less than or equal to 3400 MPa.

In one embodiment, the first material can have a tensile modulus of between of between 101 MPa and 3500 MPa measured according to ISO 527-1, and the second material can have a tensile modulus of between 0.1 MPa and 100 MPa.

In another embodiment, the first material may have a tensile modulus of between 101 MPa and 1000 MPa measured according to ISO 527-1, and the second material may have a tensile modulus of between 0.1 MPa and 100 MPa measured according to ISO 527-1.

In another embodiment, the first material can have a tensile modulus of between 101 MPa and 250 MPa measured according to ISO 527-1, and the second material can have a tensile modulus between 0.1 MPa and 100 MPa measured according to ISO 527-1.

In another embodiment, the first material can have a tensile modulus of between 50 MPa and 220 MPa measured according to ISO 527-1, and wherein the second material can have a tensile modulus between 0.1 MPa and 10 MPa measured according to ISO 527-1.

A minimum transverse cross-sectional area of the core can be at least 5 mm², at least 7 mm², at least 10 mm² or at least 14 mm².

The core can be formed of a monolithic piece of material. The monolithic piece of material, may be solid in the intermediate portion, i.e. without through-holes.

The through-holes formed in the first and second end parts can be pre-formed. This can allow the prosthesis to rotate around an anchor used to secure the prosthesis in place. A suitable anchoring system is described in US Patent Publication No. US2016235538 A1, the contents of which is incorporated herein by reference.

The core generally comprises a middle portion, a first transition portion connecting the middle portion to the first end and a second transition portion connecting the middle portion to the second end. An area of a transverse cross-section of each of the ends can be larger than a transverse cross-sectional area of the transition portion and/or the middle portion.

The middle portion can comprise a wedge shaped cross-section, tapering toward the inner edge.

The ends of the core can also be covered with cushioning material. In such examples, third and fourth through holes are formed in the cushioning material, and aligned with the first and second through-holes.

The core can consist of a single molded piece of thermoplastic material.

The core can be formed of a first polyurethane, preferably a first polycarbonate urethane, and wherein the cushioning material comprises a second polyurethane, preferably a second polycarbonate urethane.

In a second aspect of the invention there is provided a method of forming a meniscus prosthesis, the method comprising the steps of: molding a core from a first biocompatible, non-resorbable material having a first tensile modulus, wherein the core comprises an arc-shaped body. The arc-shaped body comprises a first end having a first through-hole; a second end having a second through-hole; a curved intermediate section connecting the first end and the second end. The method further comprises the step of forming a first through hole in the first end of the core and a second through hole in the second end of the core. This step can be carried out contemporaneously with the step of molding the core, or the through-holes may be cut after the core has been molded. The method further comprises the step of molding a cushioning material around at least the intermediate section of the core.

According to the method, the core can be molded as a monolithic piece. The second material may be overmolded onto the first material by injection molding.

The step of molding a cushioning material around at least the intermediate section of the core can comprise first molding the cushioning material on a first side of the core; and subsequently molding the cushioning material on a second side of the core.

In third aspect, a meniscus prosthesis comprises an arc-shaped meniscus prosthesis body having a main portion comprising a reinforcing part and two end portions comprising fixation parts. The main portion comprises a part made of a first biocompatible non-resorbable material extending between the two end portions. The reinforcing part extends between the fixation parts. The fixation parts have a through hole. The reinforcing part and the fixation parts are made of a second biocompatible, non-resorbable material. The first biocompatible, non-resorbable material has a tensile modulus of at most 100 MPa as determined by ISO 527-1 and the second biocompatible, non-resorbable material has a tensile modulus of at least 101 MPa, as determined by ISO-527-1.

The advantage of the meniscus prosthesis according to the invention is that the meniscus prosthesis is strong enough to withstand the stresses to the prosthesis after implantation and loading of the knee joint and is soft enough to prevent damage to the surrounding cartilage in the knee joint.

A further advantage is that the meniscus prosthesis is easy to implant in the knee joint. Another advantage is that the reinforcing part in the meniscus prosthesis allows fixation of the meniscus prosthesis in the knee joint. It is easy to fixate the prosthesis in the knee joint by using sutures or cables in combination with the through holes. Another advantage is that a strong and durable implant is obtained that can function for years in a human knee joint.

The meniscus prosthesis according to the invention comprises an arc-shaped prosthesis body. The prosthesis body has a main portion and two end portions. The main portion extends between the two end portions and is connected to the end portions.

The main portion of the prosthesis body comprises a part made of a first biocompatible, non-resorbable material having a tensile modulus of at most 100 MPa as determined by ISO 527-1. The tensile modulus is preferably at most 80 MPa, more preferably at most 50 MPa and most preferably at most 25 MPa. The tensile modulus of the first material is for example between 5 and 15 MPa. The tensile test according to ISO 527-1 is described in more detail in the examples.

Preferably, the first biocompatible, non-resorbable material of the main portion is a polymeric material.

The polymeric material of the main portion comprises, for example a hydrogel, for example polyvinylalcohol hydrogels, and/or a thermoplastic material, for example polyacrylonitrile polymers, elastomers, polypropylene, polyethylene, polyetheretherketones (PEEK), silicon rubbers and polyurethanes. Combinations of these thermoplastic materials can also be used.

The materials together with the design of the main portion of the meniscus prosthesis provide the required properties to the meniscus prosthesis body.

Preferably, the polymeric material used in the prosthesis body comprises a polyurethane and more preferably a polycarbonate urethane. Polycarbonate urethanes were the first biomedical polyurethanes promoted for their flexibility, strength, biostability, biocompatibility and wear resistance. These polyurethanes include, but are not limited to the following: Bionate® a polycarbonate-urethane, Bionate® II, a polyurethane with modified end groups, PurSil® a Silicone Polyether Urethane and CarboSil a Silicone Polycarbonate Urethane, Elasthane® a Polyether based Polyurethane manufactured by DSM Biomedical Inc. (“DSM”); ChronoFlex® and Hydrothane, manufactured by CARDIOTECH CTE; Tecothante® (aromatic polyether-based polyurethane), Carbothane® (aliphatic polycarbonate-based polyurethane), Tecophilic®. (aliphatic polyether-based polyurethane) and Tecoplast® (aromatic polyether-based polyurethane), manufactured by THERMEDICS; Elast-Eon®, manufactured by AorTech Biomaterials and Texin®, manufactured by Bayer Corporation. The polymeric material used in the prosthesis body can also comprise cross-linked polyurethanes. The main portion further comprises a reinforcing part made of a second biocompatible, non-resorbable material. The second biocompatible, non-resorbable material has a tensile modulus of at least 101 MPa as determined by ISO 527-1. Preferably, the tensile modulus of the second biocompatible, non-resorbable material is at most 3500 MPa, more preferably at most 3000 MPa, most preferably at most 2000 MPa. For example, the tensile modulus is between 115 and 300 MPa, preferably between 120 and 250 MPa.

Preferably, the second biocompatible, non-resorbable material is a polymeric material. The second biocompatible, non-resorbable material, for example, comprises a thermoplastic material, for example polyacrylonitrile polymers, elastomers, polypropylene, polyethylene, polyetheretherketones (PEEK), silicon rubbers and polyurethanes. Combinations of these thermoplastic materials can also be used.

More preferably the second biocompatible, non-resorbable material comprises a polyurethane and most preferably a polycarbonate urethane. The polyurethanes can be chosen from the same polyurethanes as listed for the first biocompatible, non-resorbable material.

The reinforcing part extends between the fixation parts and is connected to the fixation parts. The reinforcing part can be formed by 1 to 4 parts that are all connected to the fixation parts on both sides. The reinforcing part preferably is one monolithic part. The distance between the fixation parts, following the arc-shape of the meniscus prosthesis body, determines the length of the reinforcing part. The surface area of the reinforcing part is determined perpendicular to the plane in which the arc lies and can be chosen within wide limits by a person skilled in the art based on his technical knowledge. The surface area of the reinforcing part preferably is at least 3.5 mm², more preferably the surface area is at least 5 mm², at least 7 mm² or at least 12 mm². The reinforcing part can extend along the outer rim of the main portion. The outer rim of the meniscus is the part of the meniscus that forms the outer circumference of the arc-shaped meniscus prosthesis.

Strengthening the meniscus prosthesis has the advantage that deformation of the meniscus in the outward direction is reduced. This has the advantage that the meniscus prosthesis is stable and will be functional for prolonged periods of time when it is implanted in the knee joint.

The first and the second biocompatible, non-resorbable material can comprise additives. Examples of additives are antioxidants, processing aids, lubricants, surfactants, anti-static agents, pigments, dyes and fillers. An additive that is especially preferred is a radiopaque additive, as for example bismuth and bariumsulphate. The addition of a radiopaque additives to the first and/or the second material has the effect that the meniscus prosthesis will be visible at X-ray images of the knee joint. It this way the condition of the meniscus prosthesis after implantation can be monitored. The additives may be present in the typically effective amounts well known in the art, such as 0.001 weight % to 25 weight % based on the total amount of the first or second material.

In some embodiments the meniscus prosthesis body according to the present invention resembles the form of a native meniscus. The meniscus prosthesis body may be a meniscus prosthesis body being of a standard shape, based on a native meniscus, and available in different sizes. Such standard prosthesis may be customized to fit the patient. It may also be possible to make a copy of the patients native meniscus, e.g. with a three-dimensional (3D)-prototyping technique based on tomographic imaging techniques (e.g. CT-scans) or Magnetic Resonance Imaging. An example of a 3D-prototyping technique is rapid prototyping using for example stereo-lithographic sintering (SLS) or fused deposit modeling (FDM). In this way a meniscus body may be directly formed or a mold may be formed according to the negative image of a meniscus body of a patient. Correction of the meniscus prosthesis body or the mold after 3D-prototyping is possible to adapt the meniscus body. For example to adapt the meniscus body better to the patient needs or to amend the meniscus body to remove damage or traces of wear of the native meniscus. The mold may then be used to produce a meniscus body, e.g. with a casting, molding or hot pressing technique.

Another example of a 3D-prototyping technique is 3D-printing. An advantage of these embodiments is that it provides more comfort to the patient because once the meniscus prosthesis has been implanted and the trauma has healed, the knee joint comprising the artificial meniscus, closely resembles the knee joint with the original native meniscus. The meniscus prosthesis may behave in a similar way as the original native meniscus. An advantage of using a copy of a meniscus is that these embodiments allow a normal biomechanical motion pattern which may prevent damage of the cartilage in the knee joint. A (nearly) normal behavior of the implant in the knee may provide maximal pain relief.

The prosthesis body of the meniscus prosthesis according to the present invention further comprises two end portions. The end portions of the prosthesis body are the two portions of the prosthesis body where the arc-shaped prosthesis body ends and is narrow.

The end portions of the meniscus prosthesis body according to the present invention comprise fixation parts. As described above the fixation parts are connected to the reinforcing part. This is necessary to obtain a strong fixation of the meniscus prosthesis in the knee joint, wherein the meniscus prosthesis can withstand the forces that are applied to the knee joint during normal use. The fixation parts are made of the second biocompatible, non-resorbable material as described above. The end portion comprises a fixation part. It should be prevented that the second material of the fixation part is in contact with the cartilage in the knee joint. The second material can be a harder material and can damage the cartilage material over time in case of contact. The fixation parts can be covered with the first material. When the first material is present, preferably at least the sides of the fixation part that will come into contact with the cartilage of the femur and the tibia can be covered with the first material.

The fixation parts have a through hole. The through hole extends from one side of the fixation part to the side opposite thereof. The through hole is meant for fixation of the meniscus prosthesis in the knee joint. When first material is covering the second material of the fixation part the through hole in the fixation part can also extend through the first material.

Sutures can be provided in the through hole. In one embodiment of the invention the through hole has a first portion with a first diameter and a second portion with a second diameter larger than the first diameter. In another embodiment of the invention the through hole comprises an extended part at the side of the meniscus prosthesis that is facing the tibia plateau. The extended part of the through hole is meant to fit into a bore channel made in the tibia plateau. The extended part of the through hole can be made of the first material or of the second material and will fit into the bore channel in the tibia plateau. The extended part of the through hole will prevent damage to the suture(s) after implantation of the meniscus prosthesis by sharp edges of the bore channel in the tibia plateau.

The meniscus prosthesis can be permanently fixed in the knee joint, for example, by sutures. Sutures are preferably made from a non-resorbable material. Combinations of different sutures can be used. The suture can for example be chosen from sutures made of polymeric material like Ultra High Molecular Weight Polyethylene (UHMWPE), for example DSM Dyneema® Purity; polyamide, for example DuPont® Kevlar, Kevlar29, Kevlar49; polyvinylidene fluoride (PVDF) olyester, for example Ethibond Excel and nylon. Also sutures from other materials can be used; for example from metal like stainless-steel; titanium and nickel-titanium (Nitinol). Other suitable sutures can for example be made of ceramic material or carbon fibers. Preferably, the through holes in the meniscus prosthesis each comprise at least one suture. More preferably, the suture is a metal suture. Most preferably, the suture is a stainless-steel suture.

The sutures may be employed in a monofilament or multifilament form as a single strand or a multiple fiber twine. When more than one fiber is used in the suture the fibers can be twisted into a yarn.

Preferably, the suture is provided with a broad section at the end portion of the suture that prevents the suture from slipping through the through holes in the fixation parts. The end portion of the suture can, for example, be a knot. When the through hole in the fixation part comprises two portions with different diameters the end portion of the suture preferably has the same diameter as the portion with the largest diameter in the through hole and is provided in the portion of the through hole with the largest diameter and the main portion of the suture is provided in the portion of the through hole with the smallest diameter.

The invention is also directed to a process for the production of the meniscus prosthesis. The process comprises the following steps:

a. molding the second material to form the reinforcing part and the fixation parts;

b. making the through hole in the fixation parts; and

c. molding the first material to form the part of the main portion of the prosthesis body to enclose the reinforcing part and, optionally, the fixation parts.

Preferably, the reinforcing part and the fixation parts are molded as one piece.

Preferably, the through holes are made through the fixation parts and the first material in the end portions.

The invention is also directed to a method for replacing a native meniscus by the meniscus prosthesis according to the invention.

### BRIEF DESCRIPTION OF THE FIGURES

The invention is further illustrated by FIGS. 1-5. The dotted lines represent parts of the meniscus prosthesis that are located inside the meniscus prosthesis.

FIG. 1 is a top view of the meniscus prosthesis according to an aspect of the invention;

FIG. 2 is an isometric view of the meniscus prosthesis of FIG. 1;

FIG. 3 shows a photograph of a reinforcing part used in a test according to ISO 527-1;

FIG. 4 shows a meniscus prosthesis according to an aspect of the invention;

FIG. 5A shows the meniscus prosthesis shown in FIG. 4 divided into segments;

FIGS. 5B-5H each show a cross-sectional view of the sections shown in FIG. 5A; and

### DETAILED DESCRIPTION

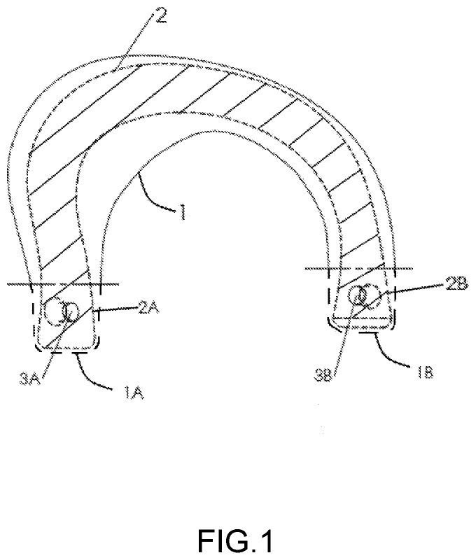

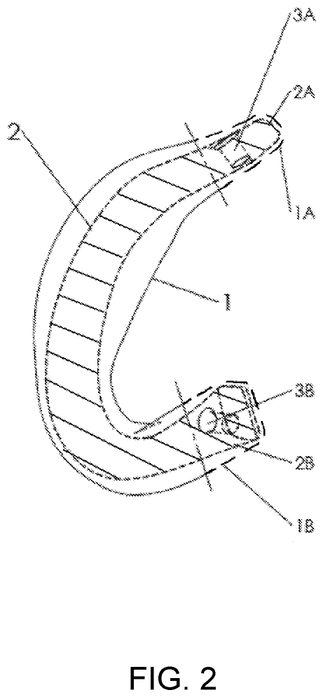

FIGS. 1 and 2 show a meniscus prosthesis according to a first aspect of the invention. As shown in FIGS. 1 and 2, the prosthesis body comprises the main portion 1 of the arc-shaped meniscus prosthesis body and two end portions 1A and 1B. The end portions 1A and 1B are configured to be secured to the bone within the joint.

The reinforcing part is represented by reference numeral 2 and the fixation parts by 2A and 2B. The reinforcing part 2 extends through the interior of the main portion 1 of the arc-shaped meniscus prosthesis body and the fixation parts 2A and 2B form at least a portion of the two end portions 1A and 1 B.

The main portion 1 and the end portions 1A and 1B are formed of a first biocompatible non-resorbable material having a first tensile modulus. The reinforcing part 2 and the fixation parts 2A and 2B are formed of a second biocompatible, non-resorbable material having a second tensile modulus.

The fixation parts 2A and 2B comprise first and second through holes 3A and 3B. As shown in FIG. 1, the first biocompatible material covers the second biocompatible material. Accordingly, to ensure that the through-holes pass all of the way through the prosthesis body, third and fourth through holes are provided in the first material, aligned respectively with the first and second through-holes 3A and 3B shown in FIGS. 1 and 2.

The first biocompatible, non-resorbable material may have a tensile modulus of at most 100 MPa as determined by ISO 527-1. The tensile modulus of the first material is preferably at most 80 MPa, more preferably at most 50 MPa and most preferably at most 25 MPa. The tensile modulus of the first material is for example between 5 and 15 MPa. The tensile test according to ISO 527-1 is described in more detail in the examples below.

The second biocompatible, non-resorbable material may have a tensile modulus of at least 101 MPa, as determined by ISO 527-1. Preferably, the tensile modulus of the second biocompatible, non-resorbable material is at most 3500 MPa, more preferably at most 3000 MPa, most preferably at most 2000 MPa. For example, the tensile modulus is between 115 and 300 MPa, preferably between 120 and 250 MPa.

### EXAMPLE 1

Prostheses for use in the knee joint must withstand repeated loading. In order to provide satisfactory patient outcomes, and avoid repeated surgical intervention, the prosthesis must also remain intact and in situ for an extended period of time, e.g. ten years or more. For prostheses comprising multiple components, separation or delamination of components from each other can be a common reason for failure of an implant.

It is therefore an object of the present disclosure to provide a meniscus prosthesis in which improved adhesion of the component parts is provided.

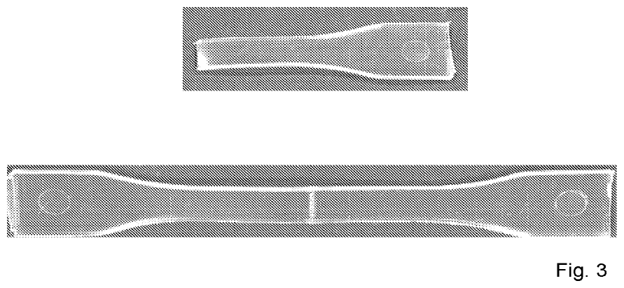

The reference sample was an injection molded 1 mm thick test specimen according to ISO 527-2 made from Bionate® II 80A. All other samples were also 1 mm in thickness but contained an adhesion interface that was created by placing half of a test specimen according to ISO 527-2 made from Bionate® II 80A in the mold prior to injection molding of the other using Bionate® II 80A under varying process conditions. These process conditions are given in Table A. In FIG. 3 the top photo is half of the tensile bar according to ISO 527-1 and the bottom photo is a tensile bar with a visible interface. Standard molding conditions for the first halves of the tensile bars were:

Melt Temperature 210° C.,

Mold temp 50° C., injection time 0.4 sec, overmolding after 5 min in environment, no preheating, melt residence time 4.4 min, holding pressure 50 MPa.

The standard molding conditions for the reference sample were:

Melt Temperature 210° C., Mold temp 50° C., injection time 0.4 sec, no preheating, melt residence time 4.4 min, holding pressure 50 MPa.

Testing was performed according to ISO-527-1. Testing was performed after annealing (24 h at 80° C. under nitrogen) and conditioning in a buffered physiological salt solution with pH 7.4 of 37 <5>C in a heated chamber kept under 70% relative humidity (RH) conditions until the samples reached a constant weight. 3-5 samples were prepared and tested for each molding condition. All samples broke at the adhesion interface. The test results are given in Table A.

TABLE A

| | | Tensile strength | Elongation at |
|---|---|---|---|
| | | (MPa) average ± | break (%) ± |
| | Molding parameters | sd | sd |
| 1 | Standard without adhesion | 17.4 ± 0.7 | 297 ± 9 |
| | interface | | |
| 2 | Standard with adhesion | 18.5 ± 0.8 | 304 ± 10 |
| | interface | | |
| 3 | 10° C. lower melt temperature | 14.9 ± 0.7 | 264 ± 11 |
| 4 | 20° C. lower melt temperature | 8.2 ± 1.2 | 96 ± 21 |
| 5 | Holding pressure 40 MPa | 22.2 ± 2.0 | 350 ± 17 |
| 6 | Holding pressure 60 MPa | 18.9 ± 4.0 | 309 ± 45 |
| 7 | Long Melt Residence time | 19.1 ± 2.0 | 346 ± 21 |
| | (4.4 →12.2 min) | | |
| 8 | Long Injection time | 19.2 ± 1.4 | 310 ± 13 |
| | (0.4 →1.2 sec) | | |
| 9 | Long storage (5 min →72 hrs) | 18.3 ± 1.6 | 332 ± 20 |
| | first half (23° C. dry, N2) | | |
| 10 | lower mold temperature | 14.9 ± 1.6 | 268 ± 23 |
| | (50 →30° C.) | | |
| 11 | preheating first half | 13.9 ± 4.0 | 267 ± 60 |
| | (23 →110° C. for 30 min) | | |

Sd = standard deviation

### Observations:

Maintaining of the above-described processing conditions for an implant with an adhesion interface led to a surprisingly strong adhesion at the interface. No loss of strength and elongation properties is observed compared to an implant without an adhesion interface.
The values for tensile strength and elongation at break of samples 1 and 2 do not show a large difference. It can thus be concluded that under standard molding conditions the presence of an adhesion interface does not make a lot of difference for tensile strength and elongation at break of a sample.
When the temperature during molding is lowered with 10 resp. 20° C. (see samples 2, 3 and 4) the tensile strength and the elongation at break of a sample becomes worse. It can be concluded that variations in the melt temperature during molding have a strong influence on the properties of the samples.
When the mold temperature is lowered from 50 to 30° C. (compare samples 2 and 10) and the mold is preheated at a temperature of 110° C. (compare samples 2 and 11) this has a clear negative influence on the tensile strength and the elongation at break of the samples.
Variations in the holding pressure (sample 5 and sample 6), melt residence time (sample 7), storing samples for 72 hrs (sample 9) and longer injection time (sample 8) have a small influence on the on the tensile strength and the elongation at break of the samples when compared with sample 2.

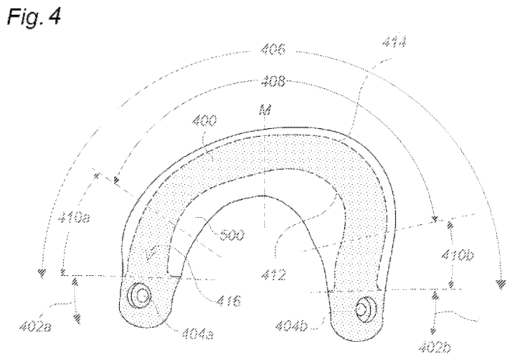

A second aspect of the invention will now be described with reference to FIG. 4. FIG. 4 shows an exemplary meniscus prosthesis. The embodiment shown in FIG. 4 is adapted to replace a native meniscus in a human knee joint. However, the skilled person will appreciate that the features and advantages described herein may be adapted for use in non-human animal joints. Moreover, the present invention is not limited application in the knee joint. Rather, the advantages described are applicable to other joints in which a native meniscus may need to be replaced.

As shown in FIG. 4, the prosthesis generally comprises a core 400 formed of a first biocompatible, non-resorbable material, and a cushioning material 500 formed of a second biocompatible, non-resorbable material, that surrounds at least a portion of the core 400 to cushion the soft tissue within the joint from the material forming the core 400. The second biocompatible, non-resorbable material is softer (e.g. has a lower tensile modulus) than the first biocompatible non-resorbable material and is intended to protect the cartilage within the joint from the harder core material. The cushioning material is therefore chosen to have a low friction interface with the cartilage, and a lower tensile modulus that the first material.

The core 400 comprises a first free end 402a having a first through-hole 404a and a second free end 402b, having a second through-hole 404b formed therein. A curved intermediate section 406 connects the first and second free ends 402a, 402b to form a generally arc-shaped body. It will be appreciated that the term arc-shaped body is used to denote a body that follows a curved trajectory. The arc-shaped body can more closely approximate a C-shape (e.g. to replace a lateral meniscus) or it may closely approximate a U-shape (e.g. to replace a medial meniscus). The arc-shaped body can comprise a simple curve (having a constant radius of curvature) or a compound curve (with a variable radius of curvature). The skilled person will understand that the precise configuration of the curve can be adapted based on the application, and may even be adjusted on a patient by patient basis.

The through-holes 404a, 404b provided in the ends 402a, 402b are configured to allow fixation of the prosthesis to a bone surface (e.g. the tibial plateau in the knee). The through-holes 404a, 404b therefore extend through the core 400 such that fixation means (e.g. a screw, staple, or suture) may be passed through the core 400 for fixation to the bone.

The shape of the core 400 is also configured to improve the reliability and longevity of the prosthesis. As shown in FIG. 4, the core 400 comprises a substantially arc-shaped curved body with a mid-point M approximately half way along the length of the curve. Although core 400 is preferably monolithic, the core 400 can be conceptually divided into five parts: a middle portion 408 approximately centred about the mid-point M of the curve; the two free ends 402a, 402b, and two transition portions 410a, 410b that connect the middle portion 408 to each of the free ends 404a, 404b. As shown in FIG. 4, together, the middle portion 408 and the transition portions 410a, 410b form the intermediate section 406 of the core 400.

The cushioning material 500 surrounds at least the intermediate section 408. The cushioning material 500 may cover only the intermediate section 408, leaving the free ends 402a, 402b exposed (as shown in FIG. 4). Alternatively, the cushioning material 500 may cover the intermediate section 406 and the free ends 402a, 402b such that the core 400 is enclosed with cushioning material 500 along its length (see e.g. FIGS. 1 and 2).

The core 400 and the cushioning material 500 perform different roles in situ, within the joint. The cushioning material 500 is configured to distribute the vertical load from the condyle to the tibial plateau, whereas the core 400 is configured to withstand the hoop stress that results from the round shape of the condyle, bearing upon the (wedge-shaped) implant and acting to extrude the implant from the joint.

The core 400 is configured to withstand the circumferential hoop stress that places the core 400 under tension. Accordingly, the core 400 can be made as a monolithic piece, e.g. a single piece of molded thermoplastic material, without joints or connections between the ends 402a, 402b, and the curved intermediate section 406. By forming the core 400 as a monolithic piece, any weak points likely to fail when repeatedly placed under tension can be minimised or eliminated. Moreover, the extension of the core 400 (due to the flexibility of the material from which it is made) can be more precisely controlled.

The core 400 is also preferably formed as a solid piece, without openings or through holes, with the exception of the first and second through-holes 404a, 404b formed in the free ends 402a, 402b of the core 400. By forming the core 400 as a solid monolithic piece, weak points likely to fail under repeated loading can be avoided.

The through-holes 404a, 404b can be pre-formed through the core 400. An advantage of providing pre-formed holes in the core 400 is that the ends 402a, 402b of the core can rotate (in a limited manner) about the fixation means that pass through the through-holes 404a, 404b to secure the prosthesis body to the bone. By allowing at least some rotation of the ends 402a, 402b about the fixation means that pass through the through holes 404a, 404b, fatigue of the material at the interface with the fixation means is reduced, thereby reducing the likelihood that the implant fails at the point of fixation.

The through-holes 404a, 404b can comprise a straight bore, or (as illustrated in FIG. 4), the through-holes 404a, 404b can comprise a stepped bore (e.g. a counterbore through-hole) to wholly or partially accommodate the head of a fastening member (e.g. a screw) or a suture knot. Such a counterbore may protect the tissue within the joint from the chosen fastener (and vice versa) and maximises the volume of the material that can be accommodated within the joint, thus increasing the durability of the ends 402a, 402b.

Referring still to FIG. 4, the core 400 comprises an inner edge 412 and an outer edge 414. The inner edge 412 of the core 400 forms the concave surface of the curve that forms the body, whilst the outer edge 414 forms the convex surface of the curve. Accordingly, in situ, the core 400 is oriented with the inner edge 412 towards the interior of the joint, whilst the outer edge 414 is oriented towards the exterior of the joint.

The core 400 also comprises an upper surface 416 and a lower surface (not shown in FIG. 4). In situ, the upper surface 416 is oriented towards the condyle, whilst the lower surface is oriented towards the tibial plateau.

In some embodiments of the invention, the core 400 comprises a flattened transverse cross-section along its length such that a maximum height Hₘₐₓ defined between the first surface and the second surface is less than a maximum width Wₘₐₓ defined between the inner edge and the outer edge of the core along the length of the core 400. By providing a flattened core 400, in-plane stiffness is high, which minimises the likelihood of in-plane buckling of the core 400 (e.g. leading to lateral dislocation or bucket handle dislocation of the implant). In a healthy native meniscus, the meniscus is fixated to its peripheral capsule and therefore cannot buckle underneath the femoral condyle. However, the meniscus prosthesis according to the disclosure is not peripherally fixated and only has two fixation points at its horns. This can avoid the need for out of joint fixation, and reduces the number of fixation points required (and thus the number of bores required in the joint).

The flattened-shape of the core 400 also reduces the out of plane stiffness of the core 400 (relative to the in-plane stiffness), which can facilitate fixation of the implant to an irregular bone surface, such as the tibial plateau.

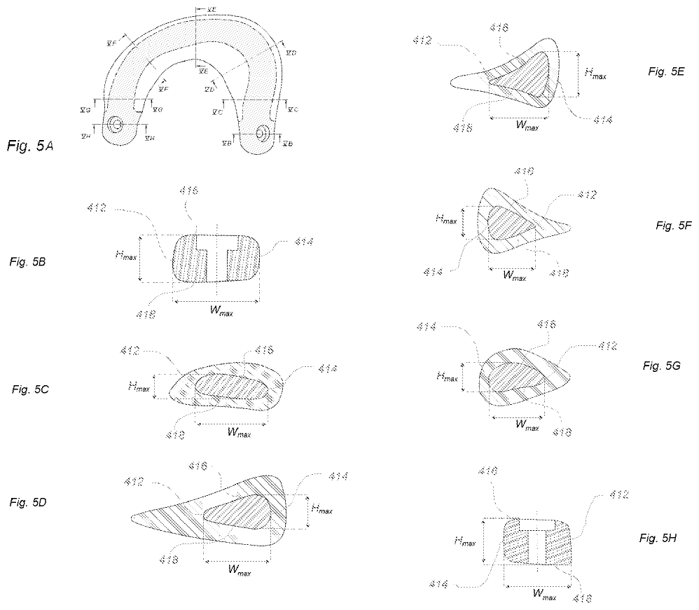

It will be understood that in the context of the present invention, “in-plane” refers to the plane in which the C-shaped curve lies. It will also be understood that the term “flattened” denotes a cross-section in which Wₘₐₓ>Hₘₐₓ. The upper and lower surfaces of the core may be substantially parallel to each other or the upper and lower surfaces may have an irregular shape, in which Wₘₐₓ>Hₘₐₓ. For example, as will be described in more detail below, the transverse cross-section may vary along the length of the core 400 and portions of the core 400 may have a tapered cross-section, in which Wₘₐₓ>Hₘₐₓ, with Hₘₐₓ is located at the outer edge 414 and Hₘᵢₙ located at the inner edge 412 form a wedge-shaped cross-section. Such an embodiment will be described in more detail with reference to FIGS. 5B-5H below.

As will be described in more detail below with reference FIGS. 5B to 5H, the transverse cross-section of the core 400 can be calculated for the middle portion 408, the free ends 404a, 404b, and the transition portions 410a, 410b to improve the function and longevity of the implant. By “transverse cross-section” it is meant a cross-section of the core taken orthogonal to the plane in which the curved body lies, and perpendicular to a tangent to the curve at a given point. The cross-sections shown in FIGS. 5B to 5H are clearly shown in FIG. 5A with lines B-B, C-C, D-D, E-E, F-F, G-G, and H-H.

FIGS. 5B and 5H show a cross-sectional view of the ends 402a, 402b. In the illustrated embodiment, the ends 402a, 402b are not covered with cushioning material 500. The upper surface 416, lower surface 418, and inner and outer edges 412, 414 are shown in FIGS. 5B and 5H.

Since the ends 402a, 402b cooperate with anchoring means to secure the implant within the joint, the dimensions of the ends 402a, 402b can be optimised to withstand wear at the interface with the anchoring means. The thickness of the material around the through-holes 404a, 404b is maximised, resulting in a high transverse cross-sectional area at the ends 402a, 402b.

FIGS. 5C and 5G each show a cross-sectional view of the transition portions 410a, 410b of the core, covered with cushioning material 500. As shown in FIGS. 5C and 5G, the transition portions 410a, 410b of the core 400 have a flattened, elongate cross-section. The flattened, elongate cross-section of the transition portions 410a, 410b can provide relatively high in-plane stiffness that prevents the implant from buckling resulting in lateral dislocation of the implant. However, the flattened shape provides increased out of plane stiffness in the transition portions 410a, 410b so that enough flexibility can be provided to allow adjustment on an irregular surface, such as the tibial plateau.

FIGS. 5D, 5E, and 5F each show a cross-sectional view of the middle portion 408 of the core 400, surrounded by cushioning material 500. As shown in FIGS. 5D-5F, the middle portion 408 of the core has a wedge shaped cross-section oriented such that the thickness of the core 400 tapers towards the inner edge 414. The wedge-shaped cross-section of the core 400 in the middle portion 408 mimics the natural shape of the meniscus. Moreover, the tapered cross-section also minimises the likelihood that the core 400 cuts through the softer cushioning material 500 if the implant is extruded from the joint. As discussed above, in at least some embodiments, the cross-section of the core 400 can be substantially constant along the intermediate portion 406, or along the entire length of the core.

As shown in FIGS. 5B to 5H, the cross-sectional area of the core 400 can vary along the length of the prosthesis. However, in order to provide a durable and reliable prosthesis, it is preferred that the core 400 has a minimum transverse cross-sectional area of at least 3.5 mm², more preferably 5 mm² more preferably at least 7 mm², more preferably at least 12 mm². By controlling the minimum cross-sectional area of the core, the risk of the core cutting through the cushioning material can be reduced. This can provide a significant improvement in the longevity of the present implant when compared to fibre or film reinforced implants because the increased transverse cross-sectional area of the core 400 minimises the cutting effect (similar to a cheese wire) of the reinforcing material through the cushioning material 500.

As shown in FIGS. 5B-5H, to increase the resilience of the ends 402a, 402b to wear and potential failure at the fixation point, the end parts are optimised for withstanding the forces applied about the anchor. Accordingly, the cross-sectional area of the ends 402a, 402b of the core are increased, relative to the cross-sectional area of the remainder of the prosthesis body, within the confines of the space available within the joint. The transverse cross-sectional area each of the end parts (through which the through-holes pass) may therefore be at least 15 mm², or at least 20 mm².

As described above, the shape and relative volumes of the first and second materials are chosen to minimise the likelihood that the prosthesis body becomes dislocated or buckles within the joint. Moreover, weak points and joints are also eliminated as far as possible. Additional advantages may also be provided by selecting the material properties of the first and second materials (i) to minimise the risk of delamination between the two materials; (ii) to minimise the risk that the first material cuts through the second material; and (iii) to allow reliable manufacturing of the prosthesis. These and other associated advantages will be described in more detail below.

As discussed above, the present invention provides a meniscus prosthesis comprising two parts, each aimed at performing a different function: a load divider, configured to distribute the vertical load from the condyle to the tibial plateau, and a retainer, configured to withstand the hoop stress through the crescent shaped prosthesis, which results from the rounded shape of the condyle.

In the embodiment described above, the core 400 acts as the retainer and the softer cushioning material 500 is the load divider. The core 400 is therefore adapted to withstand the hoop stress within the joint during loading. Moreover, to allow a degree of elongation during loading (to mimic a native meniscus and to prevent the core 400 from cutting through the cushioning material 500, the core 400 preferably experiences approximately 3% elongation at loading of 100N.

In at least one embodiment, the first biocompatible, non-resorbable material that forms the core has a tensile modulus of at most 3500 MPa. To minimise the risk of the core 400 cutting through the cushioning material, the difference between the tensile modulus of the first material and the tensile modulus of the second material is preferably at most 3400 MPa, more preferably at most 2000 MPa, more preferably at most 1000 MPa, more preferably at most 500 MPa and most preferably at most 250 MPa.

Moreover, in at least one exemplary embodiment, to prevent damage to the prosthesis and native tissue within the joint, the cushioning material 500 has a maximum tensile modulus of 100 MPa, to allow deformation during normal loading.

Therefore, according to embodiments of the invention, the cushioning material 500 comprises a material having a tensile modulus of at most 100 MPa, whilst the core material 400 has a tensile modulus that is higher than the tensile modulus of the of the cushioning material, but at most 3400 MPa higher (i.e. at most 3500 MPa).

It will be appreciated that the absolute value for the tensile modulus of the first and second materials may vary within the bounds set out above. In one exemplary embodiment, the tensile modulus of the core reinforcement may be between 50 and 200 MPa, close to native meniscus tissue. In the exemplary embodiments, the softer compression modulus of the load divider may be between 0.1-10 MPa, close to meniscus/cartilage tissue. Consequently, during physiological loading, both the softer and stiffer material will allow some lengthening in the circumferential direction.

In another exemplary embodiment, the first material has a tensile modulus of at least 101 MPa (measured according to ISO 527-1), whilst the second material has a tensile modulus of at most 100 MPa (measured according to ISO 527-1). The first material has a maximum tensile modulus of 3500 MPa.

Controlling the maximum difference between the two tensile moduli prevents the softer material from creeping around the rigid inner core. This provides an improvement over, for example, known fibre reinforced implants because in such implants the narrow fibres often having a very high tensile modulus (Kevlar yarns may have a tensile modulus of over 10.000 MPa), which tend to cut through any softer cushioning material. Over time, this allows the cushioning material to creep away from the internal fibres, eventually leading to exposure of the native soft tissue to the reinforcing fibres, and partial or complete dislocation of the softer material from its intended position within the joint.

The first non-resorbable, biocompatible material can be a polymeric material, and preferably a thermoplastic polymeric material. The polymeric material can comprise a polyurethane and more preferably a polycarbonate urethane. Polycarbonate urethanes were the first biomedical polyurethanes promoted for their flexibility, strength, biostability, biocompatibility and wear resistance. These polyurethanes include, but are not limited to the following: Bionate® a polycarbonate-urethane, Bionate® II, a polyurethane with modified end groups, PurSil® a Silicone Polyether Urethane and CarboSil® a Silicone Polycarbonate Urethane, Elasthane® a Polyether based Polyurethane manufactured by DSM Biomedical Inc. (“DSM”); ChronoFlex® and Hydrothane, manufactured by CARDIOTECH CTE; Tecothante® (aromatic polyether-based polyurethane), Carbothane® (aliphatic polycarbonate-based polyurethane), Tecophilic®. (aliphatic polyether-based polyurethane) and Tecoplast® (aromatic polyether-based polyurethane), manufactured by THERMEDICS; Elast-Eon®, manufactured by AorTech Biomaterials and Texin®, manufactured by Bayer Corporation. The polymeric material used in the prosthesis body can also comprise cross-linked polyurethanes. As an example, the first non-resorbable, biocompatible material can be Bionate 75D®.

The second non-resorbable, biocompatible material can also be a polymeric material, preferably a thermoplastic polymeric material. The polymeric material can comprise a polyurethane and more preferably a polycarbonate urethane. Suitable polyurethanes for the second material include, but are not limited to the following: Bionate® a polycarbonate-urethane, Bionate® II, a poly-carbonate urethane with modified end groups, PurSil® a Silicone Polyether Urethane and CarboSil® a Silicone Polycarbonate Urethane, Elasthane® a Polyether based Polyurethane manufactured by DSM Biomedical Inc. (“DSM”); ChronoFlex® and Hydrothane, manufactured by CARDIOTECH CTE; Tecothante® (aromatic polyether-based polyurethane), Carbothane® (aliphatic polycarbonate-based polyurethane), Tecophilic®. (aliphatic polyether-based polyurethane) and Tecoplast® (aromatic polyether-based polyurethane), manufactured by THERMEDICS; Elast-Eon®, manufactured by AorTech Biomaterials and Texin®, manufactured by Bayer Corporation. The polymeric material used in the prosthesis body can also comprise cross-linked polyurethanes. As an example, the first non-resorbable, biocompatible material can be Bionate 80A®.

The second polymeric material can also comprise a hydrogel, for example polyvinylalcohol hydrogels, and/or a thermoplastic material, for example polyacrylonitrile polymers, elastomers, polypropylene, polyethylene, polyetheretherketones (PEEK), silicon rubbers. Combinations of these thermoplastic materials can also be used.

In at least one embodiment, the first and second materials are formed of the same type of material, e.g. both the first and second material are formed of a polyurethane material. By selecting the same type of material for the first and second material, the risk of delamination between the first and second materials is reduced, since adhesion at the interface of the two materials is improved.

By selecting a thermoplastic material for the first and second materials, it is possible to form the prosthesis body by molding the core and the cushioning portion. The first material that forms the core may be formed first (e.g. by injection molding), and the second material that forms the cushioning portion may be form around the core 400 (e.g. by overmolding). Such a manufacturing method has advantages over known systems because the molded core 400 can maintain its shape and mechanical properties, even under the pressure and temperature conditions required to mold the cushioning material. This is particularly advantageous when compared to e.g. fibre or film reinforced implants, in which it can be extremely difficult to reliably maintain the reinforcing fibre(s)/film(s) in place during formation of the outer cushioning material. As an example, a fibre reinforced implant may comprise woven matrix of reinforcing fibres. The woven matrix is porous, with gaps between adjacent fibres. During molding of an outer material, the fibres are pushed together, forming a bundle or rope, which unpredictably realigns the fibres within the body and alters the material properties of the reinforcement (e.g. by reducing elasticity), thereby increasing the likelihood that the reinforcement will eventually cut through the cushioning material.

Accordingly, the present invention also provides a method of manufacturing a meniscus prosthesis. The method comprises the steps of: molding a core 400 made of a first biocompatible, non-resorbable material having a first tensile modulus, the core 400 having a first through hole in the first end of the core and a second through hole in the second end of the core. After molding the core 400, the cushioning material 500 can be molded around the core 400.

The cushioning material 500 can be overmolded over the core material 400 directly, or the second material can be overmolded over the core after cooling the first material. To ensure maximum adhesion between the first and second materials, the first material can be maintained in a dry environment or dried thoroughly before overmolding the second material.

The step of overmolding the cushioning material 500 can comprise two sub-steps, which may advantageously ensure centring of the first material 400 within the cushioning material 500. For example, in one example, the method comprises the step of molding the core 400 by injection molding the first material in a first mold. The molded core 400 can be placed in a second mold, which is used to mold the cushioning material 500. The core 400 is located in the second mold with an insert that spaces the core 400 from the walls of the second mold and maintains it in position. The insert extends around or contacts the core 400 in a first region. A second region is not in contact with the insert.

The cushioning material 500 is molded around the core 400 in the second region (free from the insert). After a suitable drying/curing time, the insert is removed, and the cushioning material 500 is molded around the core 400 in the region, thereby covering the core 400 with the cushioning material 500 around at least the intermediate portion.

The manufacturing technique described above provides material advantages over known implant systems. In particular, by molding the second material in two steps, it is possible to ensure accurate positioning of the core 400 within the cushioning material 500. This ensures that the core 400 is reliably covered with the softer material 500, to protect the soft tissue in the knee joint.

It should also be noted that the above-described two- or three-step manufacturing process may provide improved performance over known implant systems because the materials chosen for the core 400 and the softer material 500 are chemically similar, e.g. both polycarbonate urethanes, such that chemical bonding may occur at the interface of the two materials 400, 500.

The method of manufacturing may further comprise covering the end parts 402a, 402b of the core 400 with cushioning material, and providing third and fourth through-holes, aligned with the first and second through-holes, through the cushioning material. A suitable method for forming the meniscus prosthesis is described in US2013/0131805A1, the entire contents of which is hereby incorporated by reference.

Although the invention has been described in detail for purposes of illustration, it is understood that such detail is solely for that purpose and variations can be made therein by those skilled in the art without departing from the spirit and scope of the invention as defined in the claims. It is further noted that the invention relates to all possible combinations of features described herein, preferred in particular are those combinations of features that are present in the claims.

It is noted that the term ‘comprising’ does not exclude the presence of other elements. However, it is also to be understood that a description on a product comprising certain components also discloses a product consisting of these components. Similarly, it is also to be understood that a description on a process comprising certain steps also discloses a process consisting of these steps.

1. A meniscus prosthesis comprising:
a core made of a first biocompatible, non-resorbable material having a first tensile modulus, wherein the core comprises an arc-shaped body having:
a first end having a first through-hole configured to receive an anchor for securing the first end to a bone surface;
a second end having a second through-hole configured to receive an anchor for securing the second end to a bone surface; and
a curved intermediate section connecting the first end and the second end,
wherein the core further comprises:
a first surface configured to face a first interior surface of the joint during use and a second surface configured to face a second interior surface of the joint during use, wherein the first and second through-holes extend from the first surface to the second surface;
an inner edge and an outer edge; and
wherein a width W is defined between the inner edge and the outer edge of the core, and wherein a maximum height Hₘₐₓ is defined, perpendicular to the width W, between the first surface and the second surface, and
wherein the core comprises a transverse cross-section in which W is greater than H, along the length of the core;
a cushioning material surrounding the intermediate section of the core, the cushioning material being made of a second biocompatible, non-resorbable material having a second tensile modulus, which is lower than a tensile modulus of the first material.

2. The meniscus prosthesis according to claim 1, wherein a difference between the first tensile modulus and the second tensile modulus is less than or equal to 3400 MPa.

3. The meniscus prosthesis according to claim 1, wherein the first material has a tensile modulus of between of between 101 MPa and 3500 MPa measured according to ISO 527-1, and the second material has a tensile modulus of between 0.1 MPa and 100 MPa.

4. The meniscus prosthesis according to claim 1, wherein the first material has a tensile modulus of between 101 MPa and 1000 MPa measured according to ISO 527-1, and the second material has a tensile modulus of between 0.1 MPa and 100 MPa measured according to ISO 527-1.

5. The meniscus prosthesis according to claim 1, wherein the first material has a tensile modulus of between 101 MPa and 250 MPa measured according to ISO 527-1, and the second material has a tensile modulus between 0.1 MPa and 100 MPa measured according to ISO 527-1.

6. The meniscus prosthesis according to claim 1, wherein the first material has a tensile modulus of between 50 MPa and 220 MPa measured according to ISO 527-1, and wherein the second material has a tensile modulus between 0.1 MPa and 10 MPa measured according to ISO 527-1.

7. The meniscus prosthesis according to claim 1, wherein a minimum transverse cross-sectional area of the core is at least 5 mm².

8. The meniscus prosthesis according to claim 1, wherein the core is formed of a monolithic piece of material.

9. The meniscus prosthesis according to claim 1, wherein the first and second through-holes are pre-formed.

10. The meniscus prosthesis according to claim 1, wherein the first and second through-holes extend through the first and second ends respectively, from the first surface to the second surface.

11. The meniscus prosthesis according to claim 1, wherein the core comprises a middle portion, a first transition portion connecting the middle portion to the first end and a second transition portion connecting the middle portion to the second end.

12. The meniscus prosthesis according to claim 11, wherein an area of a transverse cross-section of each of the ends is larger than a transverse cross-sectional area of the transition portion and/or the middle portion.

13. The meniscus prosthesis according to claim 11, wherein the middle portion comprises a wedge shaped cross-section, tapering toward the inner edge.

14. The meniscus prosthesis according to claim 1, wherein the ends of the core are covered with cushioning material, and wherein third and fourth through holes are formed in the cushioning material, and aligned with the first and second through-holes.

15. The meniscus prosthesis according to claim 1, wherein the core is a single molded piece of thermoplastic material.

16. The meniscus prosthesis according to claim 1, wherein the core comprises a first polyurethane, preferably a first polycarbonate urethane, and wherein the cushioning material comprises a second polyurethane, preferably a second polycarbonate urethane.

17. A method of forming a meniscus prosthesis, the method comprising the steps of:
molding a core made of a first biocompatible, non-resorbable material having a first tensile modulus, wherein the core comprises an arc-shaped body having:
a first end having a first through-hole;
a second end having a second through-hole;
a curved intermediate section connecting the first end and the second end,
forming a first through hole in the first end of the core and a second through hole in the second end of the core;
molding a cushioning material around at least the intermediate section of the core.

18. The method according to claim 17, wherein the core is molded as a monolithic piece.

19. The method according to claim 17, wherein the second material is overmolded onto the first material by injection molding.

20. The method according to claim 17, wherein the step of molding a cushioning material around at least the intermediate section of the core comprises:
first molding the cushioning material on a first side of the core;
subsequently molding the cushioning material on a second side of the core.
